(19) United States
(12) Patent Application Publication
(10) Pub. No.: US 2026/0049102 A1
(43) Pub. Date: Feb. 19, 2026

(54) A PRO-MOIETY FOR FORMING A PRODRUG SELECTIVELY CLEAVED BY PROSTATE-SPECIFIC ANTIGEN (PSA)

(71) Applicant: THE UNIVERSITY OF SYDNEY, Sydney New South Wales (AU)

(72) Inventors: Trevor William HAMBLEY, Sydney, New South Wales (AU); Elisabeth TONDL, Sydney, New South Wales (AU)

(21) Appl. No.: 19/103,946

(22) PCT Filed: Jul. 11, 2023
(86) PCT No.: PCT/AU2023/050638

§ 371 Date: Feb. 14, 2025

(30) Foreign Application Priority Data
Aug. 17, 2022 (AU) 2022902340

(51) Int. Cl. C07K 7/06; A61K 47/65; A61K 49/00; A61P 35/04

(52) U.S. Cl. CPC C07K 7/06; A61K 47/65; A61K 49/00; A61P 35/04; C07K 2319/00

(57) ABSTRACT

A pro-moiety and a composition comprising a pro-moiety for use in a therapeutically or diagnostically effective amount in a method for detecting and/or treating prostate cancer in a subject is disclosed. The pro-moiety comprises a first peptide, and a second peptide that is linked to the first peptide, wherein the peptide comprises a sequence that is configured near a first terminus for conjugating to a drug to form a prodrug that is rapidly and/or highly selectively cleaved by prostate-specific antigen (PSA), and configured at a second terminus to bind with high selectivity to the active site of PSA, and the second peptide comprises a sequence having a negative charge to slow uptake of the prodrug by cells, and wherein the second peptide is cleaved from the first peptide upon proteolysis by PSA to produce a conjugate of the first peptide and the drug that is suitable for uptake by target cells.

### FIELD OF THE INVENTION

The present invention relates to a pro-moiety for forming a prodrug that is rapidly and/or selectively cleaved by prostate-specific antigen (PSA), a composition comprising a prodrug formed using said pro-moiety, and a method and use for detecting and/or treating prostate cancer in a subject by administering the composition in a therapeutically or diagnostically effective amount to the subject.

The invention has been developed primarily for use in detecting and/or treating prostate cancer, and will be described hereinafter with reference to this application. Those skilled in the art will appreciate that the invention may be applied to the treatment of other conditions, particularly those mediated by proteolytic enzymes.

### BACKGROUND OF THE INVENTION

Prostate cancer cells express extraordinarily high levels of PSA, a chymotrypsin-like serine protease also known as human kallikrein 3 (KLK3 or hK3), which is measurable in the blood and thus capable of being used as a clinical test to detect prostate cancer and follow response to therapy.

Prodrugs can incorporate a masking moiety that is removed from the actual drug component under target conditions by mechanisms such as degradation or enzyme-mediated cleavage.

In this respect, prostate cancers can be targeted for drug delivery by exploiting their expression of PSA, where a drug is released from the masking moiety when PSA cleaves the prodrug substrate. Exploiting PSA offers significant advantages for the diagnosis and treatment of prostate cancer because PSA is enzymatically active at high levels only in the extracellular fluid surrounding healthy and cancerous prostate cells, where the active form is expressed at high concentrations.

However, for this approach to work, the link between the drug-containing moiety and the masking moiety must be cleaved quickly by PSA, while remaining resistant to cleavage by other enzymes. These dual requirements for selectivity and timely activation are believed to have slowed the development of drugs for detecting and/or treating prostate cancer that have been masked in this way.

The present invention thus seeks to provide a pro-moiety for conjugating to a drug to form a prodrug that is rapidly and/or selectively cleaved by prostate-specific antigen (PSA), a composition comprising said pro-moiety conjugated to a drug to form a prodrug, and a method and use for detecting and/or treating prostate cancer in a subject by administering the composition in a therapeutically or diagnostically effective amount to the subject, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the present invention there is provided a pro-moiety, comprising:

a first peptide; and

a second peptide that is linked to the first peptide,
wherein the first peptide comprises a sequence that is configured near a first terminus for conjugating to a drug to form a prodrug that is rapidly and/or highly selectively cleaved by prostate-specific antigen (PSA), and configured at a second terminus to bind with high selectivity to the active site of PSA, and the second peptide comprises a sequence having a negative charge to slow uptake of the prodrug by cells, and
wherein the second peptide is cleaved from the first peptide upon proteolysis by PSA to produce a conjugate of the first peptide and the drug that is suitable for uptake by target cells.

According to a second aspect of the present invention there is provided a composition comprising a pro-moiety, the pro-moiety comprising:
a first peptide; and
a second peptide that is linked to the first peptide,
wherein the first peptide comprises a sequence that is configured near a first terminus for conjugating to a drug to form a prodrug that is rapidly and/or highly selectively cleaved by prostate-specific antigen (PSA), and configured at a second terminus to bind with high selectivity to the active site of PSA, and the second peptide comprises a sequence having a negative charge to slow uptake of the prodrug by cells, and
wherein the second peptide is cleaved from the first peptide upon proteolysis by PSA to produce a conjugate of the first peptide and the drug that is suitable for uptake by target cells.

In one embodiment, the first peptide sequence comprises HisSerSerLysLeuGln (HSSKLQ).

Suitably, the first terminus of the first peptide is the N-terminus.

Preferably, the second peptide comprises one or more amino acids that are negatively charged at physiological pH.

In one embodiment, the second peptide includes the sequence D-Asp-D-Glu (de).

Preferably, the second peptide is linked to the C-terminus of the first peptide via a spacer.

More preferably, the spacer is an amino acid sequence, wherein a first amino acid of the spacer is compatible with the active site of PSA.

Still more preferably, the first amino acid of the spacer is a leucine residue.

In one embodiment, the spacer comprises an amino acid sequence LeuGlyGly (LGG).

According to a third aspect of the present invention there is provided a method for detecting and/or treating prostate cancer in a subject, the method comprising administering to the subject, a therapeutically or diagnostically effective amount of a composition according to the second aspect.

In one embodiment, the composition is administered intratumorally and/or intraprostatically

In one embodiment, the composition is administered intravenously, intramuscularly, and/or subcutaneously.

In one embodiment, the subject has a localized prostate tumour.

In one embodiment, the subject has a metastatic prostate tumour.

Preferably, administration results in a reduction in prostate tumour volume.

Preferably, administration results in a reduction of a metastatic prostate tumour.

Preferably, administration results in treatment of the metastatic prostate tumour.

According to a fourth aspect of the present invention there is provided the use of a composition according to the second aspect, in the manufacture of a medicament for detecting and/or treating prostate cancer.

According to one embodiment of the present invention there is provided a pro-moiety comprising:
a first peptide; and
a second peptide that is linked to the first peptide,
wherein the first peptide comprises a sequence that is configured near a first terminus for conjugating to a drug to form a prodrug that is rapidly and/or highly selectively cleaved by a target protease, and configured at a second terminus to bind with high selectivity to the active site of the target protease, and the second peptide comprises a sequence having a negative charge to slow uptake of the prodrug by cells, and
wherein the second peptide is cleaved from the first peptide upon proteolysis by the target protease to produce a conjugate of the first peptide and the drug that is suitable for uptake by target cells.

According to another embodiment of the present invention there is provided a composition comprising a pro-moiety, the pro-moiety comprising:
a first peptide; and
a second peptide that is linked to the first peptide,
wherein the first peptide comprises a sequence that is configured near a first terminus for conjugating to a drug to form a prodrug that is rapidly and/or highly selectively cleaved by a target protease, and configured at a second terminus to bind with high selectivity to the active site of the target protease, and the second peptide comprises a sequence having a negative charge to slow uptake of the prodrug by cells, and
wherein the second peptide is cleaved from the first peptide upon proteolysis by the target protease to produce a conjugate of the first peptide and the drug that is suitable for uptake by target cells.

In a preferred embodiment, the target protease is prostate-specific antigen (PSA).

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a proof-of-concept strategy used in the development of a model prodrug comprising a pro-moiety according to a preferred embodiment of the present invention, for conjugating to a drug to form the prodrug. (a) The negatively charged sequence on the model prodrug is repelled from cell membranes, reducing uptake before cleavage by prostate-specific antigen (PSA); (b) the model prodrug interacts with the active site and the arginine patch of PSA, and is cleaved; (c) the model active drug is taken into the cell, and the residual negatively charged sequence is eliminated; (d) the model active drug is imaged in cells using fluorescence microscopy;

FIG. 2 shows a schematic representation of a model prodrug, in which the drug component of the prodrug is substituted with a fluorescent tag having a comparable structure to a drug;

FIG. 3 shows a chemical structure of the pro-moiety HSSKLQ↓LGGde for conjugating to a drug to produce a prodrug, where I represents the location of PSA cleavage (the PSA scissile bond);

FIG. 4 shows chemical structures of: (a) the peptidic sequence tag-HSSKLQ↓LGGde of a model prodrug, in which the drug component of the prodrug is substituted with either (c) a fluorescent anthraquinone (AQ) tag or (d) a luminescent rhenium (I) complex tag; and (b) the fragments (tag-HSSKLQ and LGGde) of the tag-HSSKLQ↓LGGde peptidic sequence following cleavage at the PSA scissile bond upon exposure to PSA, where ↓ represents the location of PSA cleavage (the PSA scissile bond); and

FIG. 5 shows Photodiode-Array Detection (PDA) traces (254 nm, 20-30 min) of (a) the peptidic sequence AQ-HSSKLQ↓LGGde (green, RT=27.4 min) of a model prodrug, and (b) the peptidic sequence of the corresponding AQ-HSSKLQ (blue, RT=26.2 min) fragment resulting from cleavage of the AQ-HSSKLQ↓LGGde model prodrug on incubation with PSA for 3, 6, 24 and 48 hours;

FIG. 6 shows composite images of the fluorescence and brightfield channels of DLD-1 cells (a colorectal adenocarcinoma cell line) dosed with AQ-HSSKLQ at a concentration of 50 μM, either in the absence of PSA (no PSA), or with added PSA (2 μg, 67 μmol), for 1, 4, and 24 hours [7.5×10³ cells were plated per well on a 96-well glass-bottomed plate. Scale bars represent 30 μm];

FIG. 7 shows composite images of the fluorescence and brightfield channels of DLD-1 cells dosed with AQ-HSSKLQ↓LGGde at a concentration of 50 μM, either in the absence of PSA (no PSA), or with added PSA (2 μg, 67 μmol), for 1, 4, and 24 hours [7.5×10³ cells were plated per well on a 96-well glass-bottomed plate. Scale bars represent 30 μm]; and

FIG. 8 shows the fluorescence channel, brightfield channel, and composite images of DLD-1 cells dosed with AQ-HSSKLQ↓LGGde at a concentration of 100 μM, either in the absence of PSA (no PSA), with 1 μg PSA (33 μmol), or with 2 μg PSA (67 μmol) for 48 hours [2.5×10³ cells were plated per well on a 96-well plastic-bottomed plate. Scale bars represent 30 μm. Fluorescence images were processed using a range of 20-1000 in the brightness and contrast settings].

### DETAILED DESCRIPTION OF SPECIFIC EMBODIMENTS

It should be noted in the following description that like or the same reference numerals in different embodiments denote the same or similar features.

In a general form, the present invention provides a pro-moiety for conjugating to a drug to form a prodrug, in which the pro-moiety comprises (i) a first peptide conjugated to the drug, and (ii) a second peptide linked to the first peptide, wherein the first peptide comprises a sequence that binds with high selectivity to a target protease, and the second peptide comprises a sequence having one or more negative charges to both slow uptake of the prodrug by a target cell and to increase the selectivity of binding to PSA via an association with the arginine patch, and wherein the second peptide is cleaved from the first peptide upon proteolysis by the target protease to produce a conjugate of the first peptide and the drug that is suitable for uptake by the target cell.

In a preferred embodiment, and as will be described in more detail below, the target protease is prostate-specific antigen (PSA).

For this purpose, it will be understood that:
(1) prostate-specific antigen (PSA) has a high substrate specificity for cleaving after glutamine (Gln) residues, a preference not shared by other proteases in human serum or those that have similar structures and functions to PSA,
(2) the hydrophilic HisSerSerLysLeuGln (HSSKLQ) peptidic sequence is an efficiently cleaved peptidic sequence specific to PSA, and
(3) PSA has a positively charged region near the active site (namely an arginine patch consisting of Arg36, Arg38 and Arg60 residues creating a high density of positive charges proximal to the active site).

With this in mind, the inventors have used molecular modelling to design a prodrug that is rapidly and/or highly selectively cleaved by prostate-specific antigen (PSA), comprising a pro-moiety for conjugating to a drug for the specific purpose of detecting and/or treating prostate cancer, in which the pro-moiety comprises a peptidic sequence having negative charges positioned for electrostatically interacting with the positively charged arginine patch of PSA, with the goal of increasing prodrug selectivity for PSA and/or the rate of cleavage.

### Composition

The present invention provides a composition comprising a pro-moiety for conjugating to a drug to form a prodrug, in which the drug in a preferred embodiment, is designed for detecting and/or treating prostate cancer.

Here, the prodrug comprises a peptide-based pro-moiety for conjugating to the drug, in which the peptide-based pro-moiety includes a negatively-charged sequence that serves the purpose of both electrostatically interacting with the positively charged arginine patch of prostate-specific antigen (PSA), and slowing the diffusion of the prodrug across the membrane of a cell due to charge repulsion. Only when the peptide-based pro-moiety is selectively cleaved in the presence of PSA to remove the negatively-charged sequence, can the remaining conjugate of the drug and some or all of the first peptide diffuse across the cell membrane.

The pro-moiety comprises a first peptide having a sequence that binds with high selectivity to the active site of prostate-specific antigen (PSA), and a second peptide, linked to the first peptide sequence via a spacer sequence, that includes the negatively-charged sequence.

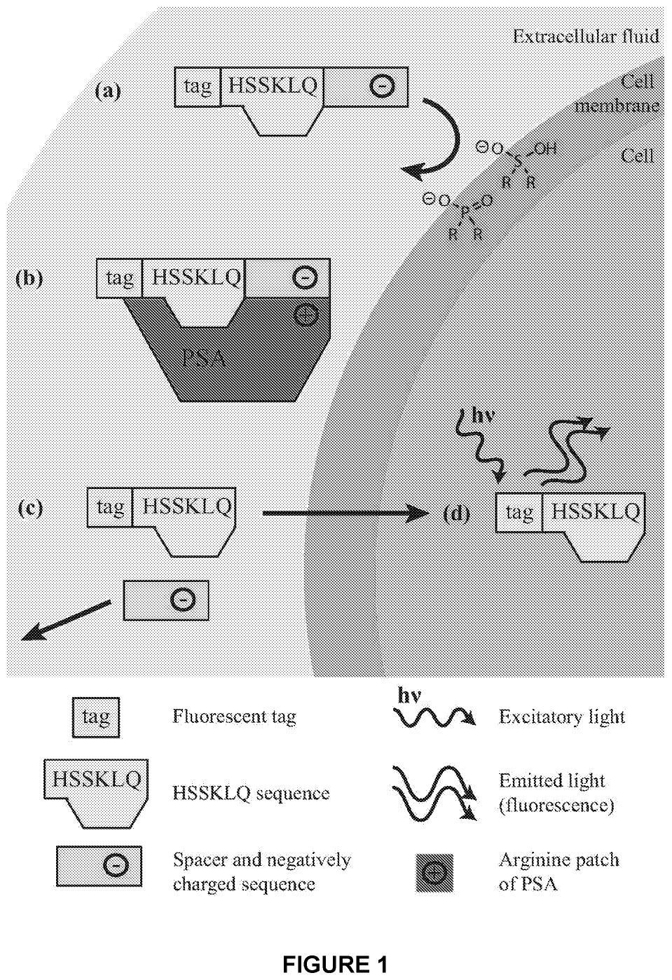

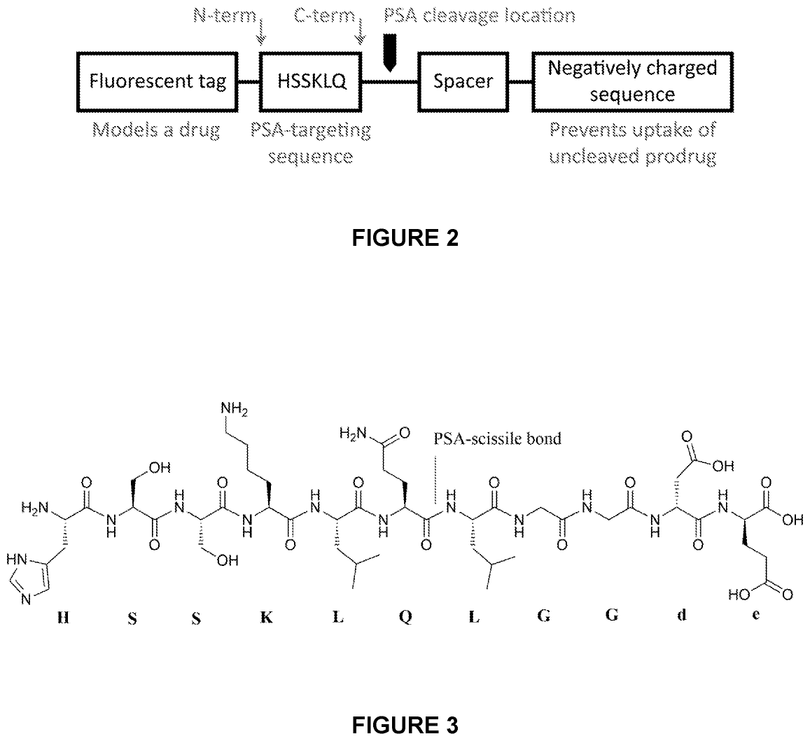

FIG. 1 shows a proof-of-concept strategy used in the development of the model prodrugs described hereinafter.

As shown in FIG. 1(a), the negatively charged sequence of the pro-moiety associated with the model prodrug is repelled from cell membranes, reducing uptake before cleavage by PSA.

FIG. 1(b) shows that when PSA is present in the extracellular fluid surrounding the target cells, the model prodrug interacts with the active site and the arginine patch of PSA, promoting the cleavage of the second peptide from the first peptide by PSA to release a conjugate of the first peptide and the drug component.

FIG. 1(c) shows that since the conjugate of the first peptide and the drug no longer carries a net negative charge, it is no longer repelled by the cell membrane and is thus able to diffuse across the cell membrane for uptake by the target cells, while the residual negatively charged sequence is eliminated.

The successful uptake of the first peptide/drug conjugate by the target cells can be confirmed by imaging using fluorescence microscopy, as possibly shown in FIG. 1(d).

### Computational Molecular Modelling

The inventors performed computational molecular modelling on certain peptide sequences to identify appropriate spacer residues and to identify appropriate negatively charged residues (e.g., Asp or Glu) for inclusion after the spacer to reduce any interference of the negative charges with the active site of PSA. The inventors also used computational molecular modelling to investigate the influence of residue chirality (D or L) on the substrate-PSA interactions.

### Peptide Sequence

Sequences with D chirality are more resistant to proteolysis than their L counterparts, [see Feng et al. and Liu et al.] and therefore comparison between the behaviour of similar sequences with different chiralities was used to establish whether there was an energy penalty associated with using D-residues.

Substrate-PSA interactions were assessed based on the number of hydrogen bonds observed between the negatively charged sequence and PSA, and on the potential energy of the system containing the substrate and PSA.

To conduct the simulations, the crystal structure of enzymatically active PSA with a substrate, LysGlyIleSerSerGlnTyr (KGISSQY), in the active site was downloaded from the Protein Data Bank (PDB: 2ZCK) [see Ménez et al.]

The KGISSQY substrate was extended at the C-terminus with the residues or sequences listed in Table 1 using the Molefacture function within Visual Molecular Dynamics software, followed by molecular dynamics and energy minimisation cycles using Nanoscale Molecular Dynamics software. [See Theoretical and Computational Biophysics Group]

TABLE 1^{#,} *
Amino acid residue sequences

| | | | | | | | | | |
|---|---|---|---|---|---|---|---|---|---|
| D | DD | DDD | E | EE | EEE | LDDD | LDDDD | LEEE | LEEEE |
| LGDDD | SGDDD | LGEEE | SGEEE | | | | | | |
| LGddd | SGddd | LGeee | SGeee | | | | | | |

^{#}Single letter amino acid residue codes are used, with uppercase indicating L chirality, and lowercase indicating D chirality.
*Abbreviations: L—Leucine (Leu), G—Glycine (Gly), D—Aspartate (Asp), E—Glutamate (Glu), S—Serine (Ser).

After energy minimisation, the potential energy of the system and the number of hydrogen bonding interactions between PSA and the negatively charged residues of the substrate were examined.

Here, the inventors found that the extended sequences interacted unfavourably with the kallikrein loop of PSA when they had zero or only one lipophilic spacer residue between the C-terminus of KGISSQY and the negatively charged residues. Interactions between the negative charges and the kallikrein loop, which locks substrates in position for cleavage, could destabilise the substrate binding and/or interfere with the enzymatic action of PSA.

Incorporating two spacer residues resulted in a lower incidence of interaction with the kallikrein loop. There was no clear energy penalty associated with incorporation of residues of D-chirality, compared with residues with L-chirality.

The inventors replaced the side chains of KGISSQY in situ to generate HSSKLQ with Q in the S1 pocket of PSA, which was subjected to energy minimisation cycles to optimise its position and geometry in the active site of PSA.

The HSSKLQ sequence was extended at the C-terminus with the sequences listed in Table 2, and the geometries obtained after further energy minimisation revealed that three-residue spacers such as SerGlyGly (SGG) and LeuGlyGly (LGG) were more effective than two-residue spacers at reducing interference with the kallikrein loop.

TABLE 2^{#, *}
Amino acid residue sequences

| | | | |
|---|---|---|---|
| LGddd | SGddd | LGeee | SGeee |
| LGGde | SGGde | LGGDE | SGGDE |

^{#}Single letter amino acid residue codes are used, with uppercase indicating L chirality, and lowercase indicating D chirality.
*Abbreviations: L—Leucine (Leu), G—Glycine (Gly), D—Aspartate (Asp), E—Glutamate (Glu), S—Serine (Ser).

Three-residue spacers followed by two negatively charged AspGlu residues of D or L chirality, were compared, and the extracted geometries did not show any interference of negatively charged side chains with the kallikrein loop.

Peptides with three residue spacers and a D-Asp-D-Glu sequence positioned the AspGlu negatively charged residues well to form hydrogen bonds and short-range electrostatic interactions. Asp and Glu residues with D-chirality formed hydrogen bonding interactions with Arg36, Arg38, and Arg60 of the arginine patch of PSA, and did not result in a significant energy penalty compared with the corresponding sequences with L chirality.

As AspAsp sequences may be challenging to synthesise, the LGGde sequence, consisting of the LGG lipophilic spacer and the de (D-Asp-D-Glu) sequence which contributes three negative charges from the side chains and C-terminus, was chosen giving HSSKLQ↓LGGde as the prodrug sequence to be investigated.

Based on this computational modelling, a pro-moiety in which the first peptide comprises the sequence HisSerSerLysLeuGln (HSSKLQ), while the second peptide includes the negatively-charged sequence D-Asp-D-Glu (de) was selected for further investigation.

### Spacer

The drug is to be conjugated to the N-terminus of the HisSerSerLysLeuGln (HSSKLQ) sequence or to an amino acid added to the N-terminal side of this sequence, while the negatively-charged D-Asp-D-Glu (de) sequence is linked to the C-terminus of the HisSerSerLysLeuGln (HSSKLQ) sequence via a spacer.

Preferably, the spacer is an amino acid sequence in which a first amino acid of the spacer is compatible with the active site of PSA such as leucine or serine.

In one embodiment, the spacer comprises a leucine residue.

In one embodiment, the spacer comprises the amino acid sequence LeuGlyGly (LGG).

The spacer sequence LeuGlyGly (LGG) was introduced to reduce interference between the negatively-charged sequence (de) and the kallikrein loop, and to allow positioning of the negative charges close to the positively charged arginine patch of PSA. Electrostatic interactions between the negative charges of the substrate and the arginine patch may increase selectivity of the prodrug for PSA, and increase cleavage rate.

FIG. 3 shows a chemical structure of the HSSKLQ↓LGGde pro-moiety for conjugating to a drug to produce a model prodrug for use in understanding the effect PSA may have on the prodrug in vitro, where I represents the location of PSA cleavage (the PSA scissile bond).

### Model Prodrugs

As will be described in more detail below, the inventors have synthesized two example prodrug models and studied the efficacy of PSA for selectively cleaving one of these model prodrugs in vitro, and in the presence of cancer cells.

For the purpose of this study, the example prodrugs comprise a fluorescent tag as a substitute for a drug component. Anthraquinone (AQ) fluorescent and rhenium (I) luminescent tags that are structurally similar to drugs and/or imaging agents were chosen for conjugation to the peptide in order to allow tracking by fluorescence microscopy in vitro.

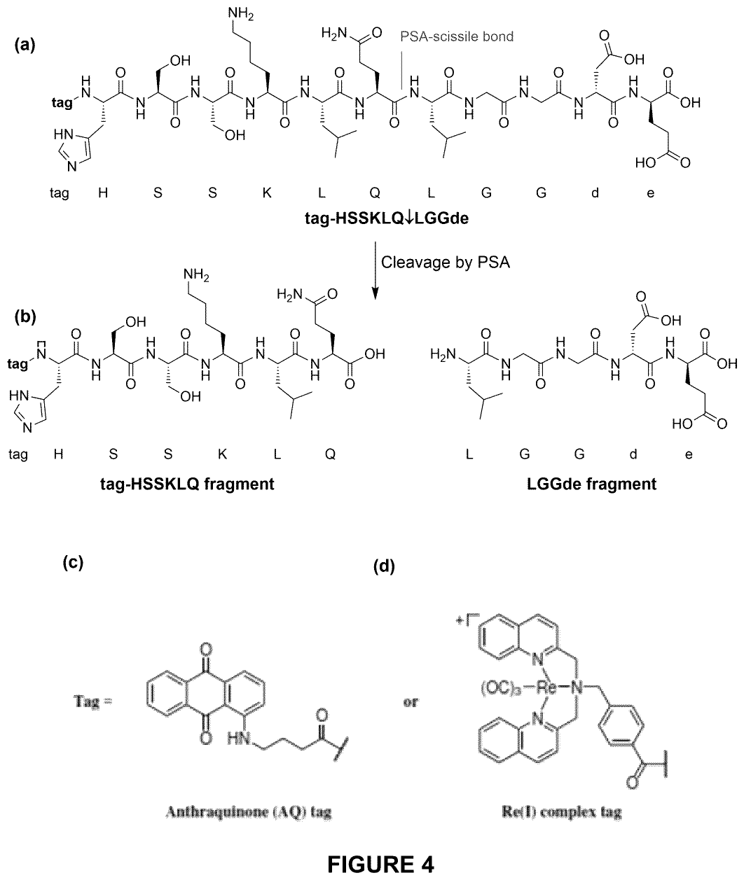

FIG. 4(a) shows a chemical structure of the peptidic sequence tag-HSSKLQ↓LGGde of a model prodrug according to a preferred embodiment of the present invention, in which the drug component is substituted with a fluorescent tag, while FIG. 4(b) shows the chemical structures of the fragments (tag-HSSKLQ and LGGde) produced following cleavage of the HSSKLQ↓LGGde peptide sequence at the PSA scissile bond upon exposure to PSA, where ↓ represents the location of PSA cleavage (the PSA scissile bond).

### Fluorescent Anthraquinone Tag

In one embodiment, the fluorescent tag selected for this purpose is structurally similar to components of established drugs and was chosen for conjugation to the peptides to enable the distribution of the conjugates to be imaged in vitro using fluorescence imaging.

FIG. 4(c) shows a chemical structure of a fluorescent anthraquinone (AQ) tag, which shares a structural similarity with such anti-neoplastic anthracycline drugs as doxorubicin and mitoxantrone. (see Kizek et al., and Hortobagyi) The AQ tag was synthesised with a carboxylic acid group to allow facile coupling to the N-terminus of the HSSKLQLGGde sequence under standard solid-phase peptide synthesis (SPPS) conditions.

### Luminescent Rhenium(I) Complex Tag

FIG. 4(d) shows a chemical structure of a luminescent Rhenium(I) complex tag with a quinolyl-based ligand. Such Re(I) complexes have garnered significant interest due to their long-lived triplet metal-to-ligand charge transfer (³MLCT) state that allows in vitro monitoring using fluorescence microscopy.

In the case of the second example, the luminescent tag is the fac-[Re(CO)₃bisquinolylamine]⁺ metal complex (see FIG. 4(d)). The stability of luminescent polypyridyl Re(I) complexes based on the facial-{M(CO)₃} core, makes them particularly suitable for imaging purposes. The fluorescent Re(I) complex tag has been conjugated to the HSSKLQ-based vector to allow comparison of PSA-mediated activation and in vitro distribution with that of the AQ-model prodrug. The Re(I) complex tag was also synthesised with a carboxylic acid linker to allow facile coupling to the N-terminus of the HSSKLQLGGde sequence under standard SPPS conditions.

### Cleavage Assays

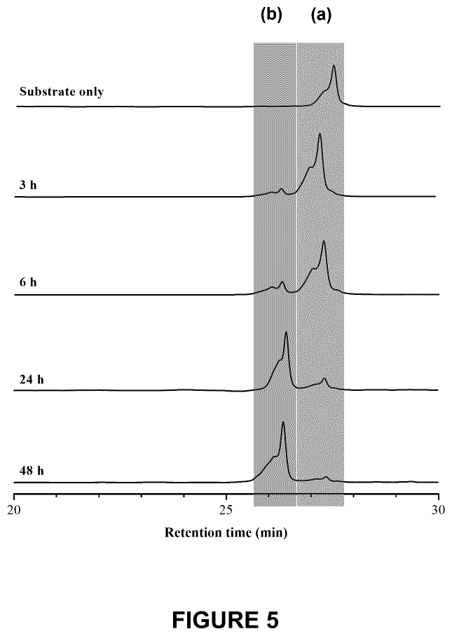

FIG. 5 shows a set of PDA traces (254 nm, 20-30 min) of (a) AQ-HSSKLQ↓LGGde (green, retention time (RT)=27.4 min), and (b) the peptide fragment, AQ-HSSKLQ (blue, RT=26.2 min), resulting from cleavage of the AQ-HSSKLQ↓LGGde model prodrug following incubation with PSA for 3, 6, 24 and 48 hours.

Incubation of AQ-HSSKLQ↓LGGde with PSA resulted in cleavage after Q and generation of the fragment corresponding to AQ-HSSKLQ, and the LGGde negatively charged sequence, which co-eluted with a persistent solvent contaminant (15.7 min, not shown). Here, the intensity (a.u.) of the uncleaved AQ-HSSKLQ↓LGGde sequence (green, RT=27.4 min) was observed to decrease over time, while the intensity of the cleaved AQ-HSSKLQ fragment (blue, RT=26.2 min) increased.

The normalised integrals of the peaks in the UV trace (254 nm) corresponding to AQ-HSSKLQ↓LGGde and the fragment equivalent to AQ-HSSKLQ in Table 3 show that AQ-HSSKLQ↓LGGde, which is in a 200-fold excess to PSA, continued to be cleaved by PSA for up to 48 hours, but that cleavage was still incomplete at that time.

TABLE 3
Normalised integrals of the AQ-HSSKLQ fragment cleaved from
AQ-HSSKLQ↓LGGde by PSA over time. Standard errors are
given where the experiment was performed in triplicate.

| | % AQ-HSSKLQ | % AQ-HSSKLQ↓LGGde |
|---|---|---|
| Time | (cleaved fragment) | (uncleaved prodrug) |
| 3 h | 11% | 89% |
| 6 h | 20% | 80% |
| 24 h | 77 (±2)% | 23 (±2)% |
| 48 h | 87 (±2)% | 13 (±2)% |

As is apparent from the data in Table 3, cleavage of AQ-HSSKLQ↓LGGde occurred at a higher rate than previously reported for a related substrate (Di Marco et al.), where 23% remained uncleaved after 24 hours' incubation. The inventors believe that the negatively charged LGGde sequence might aid substrate positioning for cleavage, allowing for a greater rate of cleavage. It is also believed that this same sequence (LGGde) might continue to interact with the arginine patch of PSA after cleavage, thereby resulting in a slowing of the rate of binding and cleavage of subsequent substrates.

### Fluorescence Microscopy

Cell imaging studies were designed to compare the effect of the absence or presence of PSA on the distribution of the tag-peptide conjugates in DLD-1 cells, which are colorectal cancer cells. The DLD-1 cell line was chosen explicitly because the cells do not express PSA, thereby allowing a controlled comparison between cells dosed with a substrate in the absence of PSA (PSA−), and cells dosed with the substrate in the presence of exogenous PSA of known concentration and uniform activity (PSA+).

For these studies, the use of human-derived prostate cancer cell lines would not have allowed a comparative model based on PSA expression, as the expression of PSA by prostate cancer cell lines varies as a function of culturing conditions, and the PSA that is expressed tends to have low levels of enzymatic activity.

The inventors predicted that in the presence of PSA, the AQ-HSSKLQ↓LGGde model prodrug would be cleaved, generating AQ-HSSKLQ for uptake by cells; but that in the absence of PSA, the model prodrug would remain uncleaved, so dosed cells would exhibit background levels of fluorescence.

It was also predicted that cells would exhibit fluorescence when dosed with AQ-HSSKLQ, regardless of the presence of PSA.

Cells were incubated with AQ-HSSKLQ↓LGGde or AQ-HSSKLQ at a concentration of 50 μM, in the absence or presence of exogenous PSA (20 μg/mL), for 1, 4, or 24 hours, unless otherwise specified.

Dosed cells were washed, then imaged using confocal fluorescence microscopy to assess conjugate distribution in the cells. Emission from the conjugated AQ tag was monitored over the range of 570-670 nm. Cell images were processed using a range of 20-300 in the brightness and contrast settings unless otherwise specified.

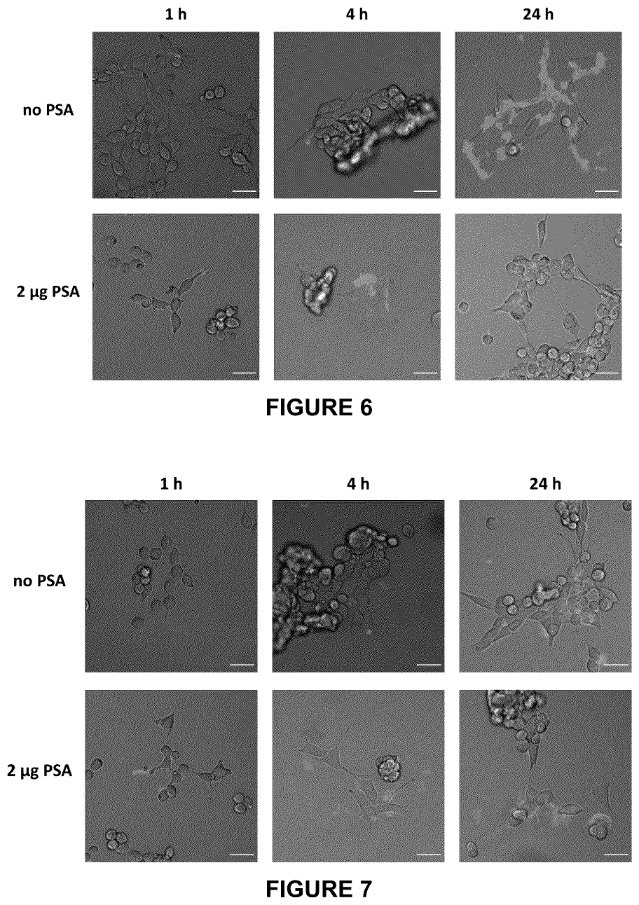

FIG. 6 shows composite images of the fluorescence and brightfield channels of DLD-1 cells dosed with AQ-HSSKLQ at a concentration of 50 μM, either in the absence of PSA (no PSA), or with added PSA (2 μg, 67 μmol), for 1, 4, and 24 hours [7.5×10³ cells were plated per well on a 96-well glass-bottomed plate. Scale bars represent 30 μm].

In cells dosed with AQ-HSSKLQ, a significant level of fluorescent precipitate was observed, and the amount of precipitate increased over time regardless of the presence of PSA, with little or no evidence of cell uptake even after incubation for 24 hours (FIG. 6). Significantly more precipitate appeared to form when cells were dosed with AQ-HSSKLQ in the presence of PSA for 1 and 4 hours, compared to cells dosed in the absence of PSA; but similar levels of fluorescent precipitate formed for the PSA+ and PSA− conditions after 24 hours' incubation.

The observed increase in precipitate formation over time could reflect the time interval required for the precipitate to form in vitro. AQ-HSSKLQ may have been taken up by cells, and the AQ tag or variants may have been expelled by exocytosis to form a precipitate, on a time scale that is too short to be measured by these studies. Formation of precipitates of AQ-HSSKLQ or its degradation products is likely to have inhibited entry of the fluorescent compounds into cells by passive diffusion and active uptake, contributing to the low levels of intracellular fluorescence.

FIG. 7 shows composite images of the fluorescence and brightfield channels of DLD-1 cells dosed with AQ-HSSKLQ↓LGGde at a concentration of 50 μM, either in the absence of PSA (no PSA), or with added PSA (2 μg, 67 μmol), for 1, 4, and 24 hours.

These images of cells dosed with AQ-HSSKLQ↓LGGde in the absence of PSA for 1, 4, and 24 hours showed that a smaller amount of fluorescent precipitate formed, and in smaller particulates, compared with images of cells that were dosed with AQ-HSSKLQ (FIG. 7). The extent of precipitation in cells dosed with AQ-HSSKLQ↓LGGde in the absence of PSA increased over time, but remained at lower levels than those observed in cells dosed with AQ-HSSKLQ, demonstrating the utility of the LGGde sequence for improving solubility.

Background levels of intracellular fluorescence indicated that AQ-HSSKLQ↓LGGde was not taken into cells in the absence of PSA. This supported the incorporation of amino acids with D chirality to increase resistance of AQ-HSSKLQ↓LGGde to clipping of the sequence by other proteases.

The images of cells dosed with AQ-HSSKLQ↓LGGde in the presence of exogenous PSA show that a significantly higher amount of precipitate formed at earlier incubation time points (1 and 4 hours) than seen in images collected in the absence of PSA, consistent with cleavage of AQ-HSSKLQ↓LGGde in vitro to yield AQ-HSSKLQ, followed by the formation of fluorescent precipitate.

In cells dosed with AQ-HSSKLQ↓LGGde and exogenous PSA, the amount of precipitate formed increased over time, consistent with ongoing generation of AQ-HSSKLQ when AQ-HSSKLQ↓LGGde is cleaved by PSA.

The extent of precipitation observed in cells incubated with AQ-HSSKLQ↓LGGde for 24 hours in the presence or absence of PSA cannot be readily distinguished based on these data alone.

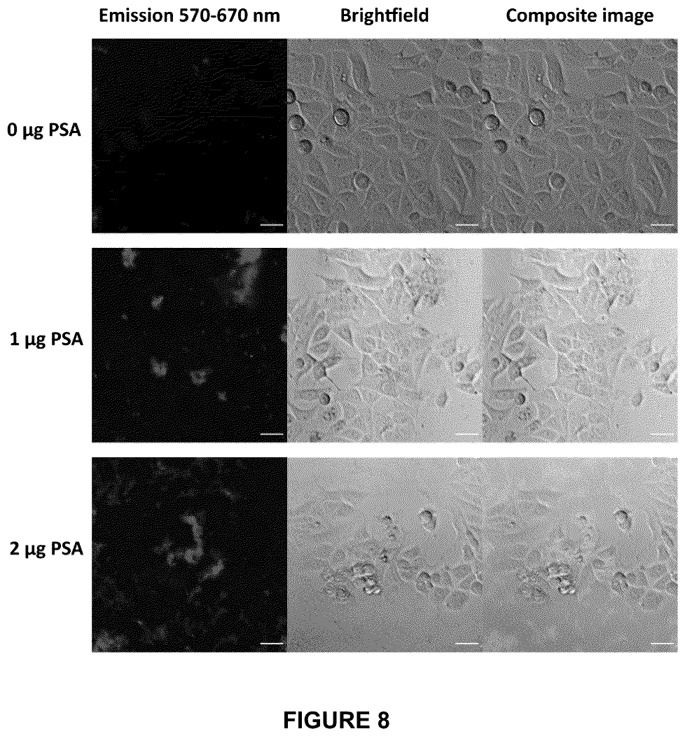

As negligible levels of intracellular fluorescence were observed after incubation for 24 hours of AQ-HSSKLQ↓LGGde in the presence of exogenous PSA, cells were incubated with AQ-HSSKLQ↓LGGde at a concentration of 100 μM and with 0, 1 (10 μg/mL) of PSA, or 2 μg (20 μg/mL) of PSA per well for 48 hours (FIG. 8).

FIG. 8 shows the fluorescence channel, brightfield channel, and composite images of DLD-1 cells dosed with AQ-HSSKLQ↓LGGde at a concentration of 100 μM, either in the absence of PSA (no PSA), with 1 μg PSA (33 μmol), or with 2 μg PSA (67 μmol) for 48 hours.

The brightness and contrast settings were adjusted during image processing to avoid washing out the emission signal with background noise. Cells incubated with AQ-HSSKLQ↓LGGde without added PSA for 48 hours showed background intracellular fluorescence, indicating that the prodrug is not taken into cells even after an extended incubation time.

Co-incubation of the cells with AQ-HSSKLQ↓LGGde and 1 or 2 μg of PSA resulted in the generation of fluorescent precipitate, indicating that PSA cleaves AQ-HSSKLQ↓LGGde in vitro to generate AQ-HSSKLQ, followed by precipitate formation, with the amount of precipitate formed increasing with increasing concentration of PSA. We observed some indications of intracellular fluorescence in cells dosed with AQ-HSSKLQ↓LGGde and 2 μg of PSA after 48 hours, suggesting that some fluorescent compound might have entered cells over the extended incubation time.

Proteolytic degradation of AQ-HSSKLQ, the fragment generated on cleavage of AQ-HSSKLQ↓LGGde, may cause the generation of insoluble sequences that readily precipitate. The poor solubility of the AQ tag is likely to have significantly influenced precipitate formation observed in vitro. The extended planar structure of the AQ tag readily allows intermolecular 90-90 stacking that would promote precipitation in aqueous environments. This is consistent with the observation of some precipitate after 48 hours' incubation of AQ-HSSKLQ with Tris buffer in the PSA cleavage assays.

The charges present on the AQ-HSSKLQ fragment may also have contributed to the poor cellular uptake.

At physiological pH, the AQ-HSSKLQ fragment has a negatively charged C-terminus and a positively charged side chain on the Lys residue, yielding no net charge. The L chirality of the HSSKLQ sequence, necessary for correct interaction with PSA to enable cleavage, renders the HSSKLQ sequence in the AQ tag vulnerable to proteolytic degradation in vitro and in vivo.

Proteases present in the media or expressed extracellularly by the DLD-1 cells may have clipped the AQ tag at any of the amide bonds present to yield a less soluble sequence such as AQ-HS or AQ-HSS with a net charge of −1, that would be electrostatically repelled from cell membranes.

Smaller and fewer particles of precipitate were observed in the images of cells dosed with AQ-HSSKLQ↓LGGde compared with those observed in the images of cells dosed with AQ-HSSKLQ, regardless of the presence of PSA.

The negative charges of the D-Asp-D-Glu sequence in AQ-HSSKLQ↓LGGde are likely to confer greater hydrophilicity, thereby increasing the solubility, and are also likely to discourage π-π stacking interactions that could promote precipitation.

In the absence of PSA, AQ-HSSKLQ↓LGGde appears to undergo little or no degradation by proteases that may be expressed by DLD-1 cells or that may be present in the supplemented media. Proteolytic degradation involving the removal of the AspGlu sequence would generate less soluble sequences such as AQ-HSSKLQ↓LGG or shorter sequences similar to AQ-HSSKLQ, which would be expected to form a significant amount of precipitate. This in vitro resistance to proteolysis is likely to be enhanced by the D chirality of the C-terminal AspGlu sequence of AQ-HSSKLQ↓LGGde.

While the formation of in vitro fluorescent precipitate in these studies is disappointing and complicate the interpretation, these results do suggest that AQ-HSSKLQ↓LGGde was successfully cleaved by PSA in the presence of cancer cells, presumably generating AQ-HSSKLQ, followed by the formation of precipitate. The increase in the amount of precipitate formed in cells dosed with AQ-HSSKLQ↓LGGde in the presence of PSA over time indicates that PSA retained its enzymatic activity and continued to cleave substrates over time.

### Method of Treatment

The present invention also provides a method and use of the composition described above in the manufacture of a medicament for detecting and/or treating prostate cancer in a subject.

The composition comprises a prodrug formed using a drug component, that is, an active pharmaceutical ingredient (API), in place of the AQ fluorescent tag described above, which can be administered in a therapeutically or diagnostically effective amount to any subject, including a human or non-human animal, in an amount effective to treat a disorder.

These prodrugs can be administered parenterally by injection or by gradual infusion over time. The prodrugs can be administered intratumorally, intraprostatically, intravenously, intraperitoneally, intramuscularly, subcutaneously, intracavity, or transdermally.

Other methods of administration will be known to those skilled in the art.

In one embodiment, the prodrug composition is administered in a therapeutically or diagnostically effective amount to a subject with a localized or metastatic prostate tumour for the treatment thereof, where said treatment is for the purpose of reducing the volume or size of the tumour.

Preparations for parenteral administration of a prodrug of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases.

### Definitions

As used herein, the term “prodrug” means compounds that are drug precursors which, following administration to a subject, release the drug in vivo via some chemical or physiological process (e.g., a prodrug on being brought to the physiological pH or through enzyme action is converted to the desired active drug form). The prodrug can be converted into a product that is toxic to tumour cells.

Persons of skill in the relevant art will understand that the terms “drug” and “prodrug” used herein, may include diagnostic agents and pro-diagnostic agents, respectively.

As used herein, the term “pro-moiety” refers to the functional part of the prodrug that is used to modify the structure of the drug to improve the physicochemical, biopharmaceutical and/or pharmacokinetic properties of the drug.

As used herein, the term “prostate specific antigen” (PSA) means prostate specific antigen, also known as human kallikrein 3 (KLK3 or hK3), as well as all other proteases that have the same or substantially the same proteolytic cleavage specificity as prostate specific antigen.

As written herein, amino acid sequences are presented according to the standard convention, namely that the amino terminus of the peptide is on the left, and the carboxy terminus on the right.

As used herein the term “therapeutically or diagnostically effective amount” refers to the amount of a medicament or a pharmaceutically active ingredient that is delivered to a subject to provide the desired physiological response. Methods for preparing pharmaceutical compositions are within the skill in the art, for example as described in Remington's Pharmaceutical Science, 18^{th} ed., Mack Publishing Company, Easton, Pa. (1990), and Remington: the Science and Practice of Pharmacy, 20^{th} ed., Lippincott Williams & Wilkins, (2003).

As used herein the term “administering” or “administration” as used herein means the introduction of a foreign molecule into a cell or host. The term is intended to be synonymous with the term “delivery”.

As used herein the term “treating” or the phrase “to treat” refers to any type of treatment that imparts a benefit to a subject afflicted with a disease, including improvement in the condition of the subject (e.g., in one or more symptoms), delay in the progression of the condition.

As used herein, the term “about” as used herein when referring to a measurable value such as a parameter, an amount, a temporal duration, and the like, is meant to encompass variations of ±10% or less, preferably ±−5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

As used herein, the singular forms “a”, “an”, and “the” include both singular and plural referents unless the context clearly dictates otherwise.

As used herein, the terms “amino acid” and “peptide” are understood to include groups that can be incorporated in a peptidic sequence such that it retains the ability to bind to the active site of a protease and be cleaved by that protease.

As used herein, the residues of a peptidic amino acid sequence with L-chirality are written in upper-case, while the residues with D-chirality are written in lower-case (e.g., LGGde).

### Materials and Methods

### Synthesis of the AQ Tag

This method was adapted from Di Marco et al. (2019).

During synthesis and work-up, the reaction mixtures and product were protected from light. 1-Chloroanthraquinone (2.64 g, 10.9 mmol, 1.1 eq.) was combined with dry DMA (60 mL) and TEA (3.0 mL, 21.4 mmol, 2.2 eq.). 4-Aminobutyric acid (1.04 g, 10.1 mmol, 1.0 eq.) was added and the reaction mixture was heated to 160° C. for 5 hours. The reaction mixture was cooled to 55° C. and was concentrated overnight under a stream of nitrogen gas. The mixture was acidified with a solution of 0.1 M HCl_{(aq)} (60 mL) and extracted into DCM (2×80 mL), then washed with water (2 ×60 mL) and extracted into the aqueous layer using a solution of 0.1 M NaOH (aq) (2×60 mL). The aqueous layer was washed with DCM until the organic layer was clear, and the aqueous layer was then acidified with a solution of 1 M HCl_{(aq)} to precipitate the product. The product was collected by vacuum filtration as a dark red solid (0.19 g, 0.601 mmol, 6% yield).

¹H NMR (400 MHz, MeOD-d⁴, ppm): δ 8.27 (m, 2H), 7.85 (ddd, J=7.5 Hz, J=1.5 Hz, 2H), 7.64 (dd, J=7.4 Hz, J=8.6 Hz, 1H), 7.29 (dd, J=0.7 Hz, J=8.6 Hz, 1H), 3.47 (t, J=7.2 Hz, 2H), 2.51 (t, J=7.2 Hz, 2H), 2.04 (quintet, J=7.2 Hz, 2H).

¹³C NMR (75 MHZ, DMSO-d⁶, ppm) δ 183.96, 182.79, 173.97, 151.28, 135.57, 134.41, 134.30, 133.92, 133.38, 132.31, 126.36, 126.19, 118.49, 115.00, 112.00, 41.45, 30.99, 24.06.

LRMS-ESI (−) m/z: [M−H]⁻ found 308.25, calc. 308.09 for C₁₈H₁₄NO₄⁻.

### Methods for the Synthesis of the Re(I) Complex Tag

The methods for the syntheses below are adapted from Green (2013) unless otherwise specified.

### Synthesis of Methyl Ester Quinolyl Ligand

TEA was dried for 48 hours over anhydrous sodium sulfate. Methyl 4-(aminomethyl)benzoate hydrochloride (0.61 g, 3.03 mmol, 1.0 eq.) was combined with dry MeCN (10 mL), and dry TEA (0.42 mL, 3.03 mmol 1.0 eq.) to neutralise the mixture. 2-(Chloromethyl)quinoline hydrochloride (1.43 g, 6.65 mmol, 2.2 eq.) was combined with dry MeCN (10 mL), and dry TEA (0.93 mL, 6.67 mmol, 2.2 eq.) to neutralise the mixture. Both mixtures were sonicated to aid the formation of fine particulates and were combined under inert conditions. A further 2.2 eq. of dry TEA (0.93 mL, 6.67 mmol) was added, and the reaction mixture was refluxed for 24 hours under an inert atmosphere. The mixture was cooled to room temperature and concentrated under a stream of nitrogen gas, then diluted with a saturated NaHCO_{3(aq)} solution (20 mL). The product was extracted into chloroform (4×20 mL) and washed with brine (3×15 mL), then dried over anhydrous sodium sulfate. The mixture was filtered, and the solvent was removed from the crude product under reduced pressure. The product was purified by column chromatography without flashing on a silica stationary phase using 10-55% EtOAc in hexane as the eluent. Solvents from the column were removed under reduced pressure to yield a golden-brown solid (0.71 g, 1.60 mmol, 53% yield).

¹H NMR (400 MHZ, CDCl₃-d¹, ppm): δ 8.14 (d, J=8.5 Hz, 2H), 8.05 (d, J=8.5 Hz, 2H), 7.99 (d, J=8.1 Hz, 2H), 7.78 (d, J=8.1 Hz, 2H), 7.69 (m, 4H), 7.51 (m, 4H), 4.01 (s, 4H), 3.89 (s, 3H), 3.81 (s, 2H).

¹³C NMR (75 MHZ, DMSO-d⁶, ppm) δ 166.95, 159.92, 147.50, 144.49, 136.48, 129.66, 129.46, 128.99, 128.96, 127.51, 127.35, 126.25, 120.95, 60.93, 58.46, 52.02.

LRMS-ESI (+) m/z: [M+H]⁺ found _{448.17}, calc. _{448.20} for C₂₉H₂₆N₃O₂⁺. R_{f}=0.23 in a 50% mixture of EtOAc in hexane on a silica plate.

### Synthesis of Quinolyl Ligand

Methyl-4-((bis(quinolin-2-ylmethyl)amino)methyl)benzoate (0.47 g, 1.05 mmol) was dissolved in a 1:1 mixture of MeCN and 1 M KOH_{(aq)} and heated at 95° C. for 4 hours with stirring. After cooling to room temperature, the reaction mixture was concentrated under a stream of nitrogen gas, then diluted with water (10 mL). The acidity of the mixture was adjusted to pH 4 using a 1 M solution of HCl_{(aq)} to precipitate the product, and the product was extracted into chloroform (6×15 mL) while the acidity of the aqueous layer was maintained at pH 4. The chloroform was removed under reduced pressure to yield the product as a golden-brown solid (0.39 g, 0.91 mmol, 85%).

¹H NMR (400 MHZ, DMSO-d⁶, ppm): δ 8.35 (d, J=8.5 Hz, 2H), 7.94 (m, 6H), 7.74 (m, 4H), 7.57 (m, 4H), 3.93 (s, 4H), 3.77 (s, 2H).

¹³C NMR (75 MHZ, MeOD, ppm) δ 169.77, 161.09, 148.09, 145.09, 138.56, 131.00, 130.83, 130.20, 128.90, 128.77, 127.72, 122.43, 79.44, 61.80, 59.99.

LRMS-ESI (−) m/z: [M−H]⁻ found 432.18, calc. 432.17 for C₂₈H₂₂N₃O₂⁺. R_{f}=0.00 in a 50% mixture of ethyl acetate in hexane on a silica plate.

### Synthesis of Triaquatricarbonylrhenium(I) Bromide

This procedure was adapted from Lazarova et al. (2004).

Water (25 mL) was added to bromopentacarbonylrhenium(I) (3.33 g, 8.19 mmol) and was heated to reflux. During the reaction time, the condenser was periodically flushed with water. After 24 hours had elapsed, the reaction mixture was cooled to room temperature and filtered to remove remaining starting material. The water was removed from the product under reduced pressure, yielding a white-green coloured solid, identified as triaquatricarbonylrhenium(I) bromide (1.44 g, 3.55 mmol, 43% yield). The product was used without further purification in the subsequent complexation reaction.

### Synthesis of Re(I) Complex Tag

4-((Bis(quinolin-2-yl)amino)-methyl)benzoic acid (0.33 g, 0.763 mmol, 1.0 eq.) and triaquatricarbonylrhenium(I) bromide (0.45 g, 1.11 mmol, 1.5 eq.) were combined in MeOH. The acidity of the mixture was adjusted to pH 8 using 0.5 M NaOH_{(aq)} and the mixture was refluxed for 4 hours. The reaction mixture was concentrated under a stream of nitrogen gas and extracted into DCM (15 mL) then washed twice with water (2×10 mL). The DCM was removed from the reaction mixture under reduced pressure and the crude product was dissolved in 3:1 MeOH:MeCN and purified by preparative HPLC using a gradient of 30-60% unbuffered MeCN in 0.1% w/v NH₄OAc buffered milliQ water over 30 minutes (RT=21.3 min). Fractions containing the pure product were combined and residual buffer was removed by sequential lyophilisation. Purity of the product was ascertained by LCMS, performed with a gradient of 0-50% buffered MeCN over 30 minutes (RT=21.0 min). The purified product was concentrated under nitrogen gas, then lyophilised twice to remove residual NH₄OAc, and collected as a light brown solid (0.30 g, 0.382 mmol, 50%).

¹H NMR (400 MHZ, MeOD-d⁴, ppm) δ 8.55 (d, J=9.2 Hz, 2H), 8.47 (d, J=8.4 Hz, 2H), 8.14 (d, J=8.2 Hz, 2H), 7.97 (d, J=8.1 Hz, 2H), 7.86 (t, J=8.0 Hz, 2H), 7.78 (d, J=8.2 Hz, 2H), 7.68 (t, J=7.2 Hz, 2H), 7.55 (d, J=8.5 Hz, 2H), 5.46 (d, 2J=17.4 Hz, 2h), 5.17 (s, 2H), 4.73 (d, 2J=17.2 Hz, 2H).

¹³C NMR (100 MHZ, MeOD, ppm) δ 166.04, 163.04, 148.16, 142.82, 140.24, 137.16, 134.73, 133.98, 132.96, 131.07, 130.76, 129.71, 129.53, 129.40, 121.07, 71.93, 68.77.

LRMS-ESI (+) m/z: [M]⁺ found 703.96, calc. 704.12 for C₃₁H₂₃N₃O₅Re⁻.

HRMS (+) m/z: [M]⁺ found 404.1194, calc. 704.1190 for C₃₁H₂₃N₃O₅Re⁺.

IR: Vₘₐₓ (cm⁻¹)=2025, 1916 (fac-[Re(CO)₃]⁺); R_{f}=0.00 in a 50% mixture of EtOAc in hexane on a silica plate.

### Peptide Synthesis

Peptides were synthesised manually using loaded solid-phase resin supports and the Fmoc protecting group strategy in fritted syringes. The side chains of His, Ser, Lys, Gln, D-Asp, and D-Glu were protected with Trt, tBu, Boc, Trt, OtBu, and OtBu protecting groups respectively. Quantities of reagents and solvents are specified as equivalents for 0.1 g of the loaded resin, given the resin loading.

Between each step, the resin was washed with 5×DMF, 5×DCM, then 5×DMF, where individual washes were approximately 1.5 mL in volume. HSSKLQ was synthesised using commercially available Fmoc-Gln(Trt)-Wang resin (loading: 0.52 mmol g⁻¹), while HSSKLQL was synthesised using commercially available Fmoc-Leu-Wang resin (loading: 0.66 mmol g). Ctc resin was manually loaded with Fmoc-D-Glu(OtBu)-OH for the synthesis of HSSKLQLGGde.

To load the ctc resin, 0.1 g of resin was swollen in DCM and 30 μL of thionyl chloride was added slowly followed by 0.5 mL of DMF. The vessel was capped and shaken for 4 hours, then the DMF solution was expelled, and the resin washed with DCM (5×3 mL). Immediately, a freshly prepared solution of Fmoc-D-Glu(OtBu)-OH (28 mg, 66 μmol, 1.1 eq.) and DIPEA (17 μL, 0.18 mmol, 3.0 eq.) in DCM (1.0 mL) was added and the vessel was capped and shaken for 45 min. The solution was expelled, and a fresh identical solution was added, and the vessel shaken for 16 hours.

The loaded resin was washed then used for peptide synthesis. This procedure resulted in a loading of 1.2 mmol g⁻¹, calculated from the absorbance of the dibenzofulvene-piperidine adduct at 301 nm, and was chosen to offset the short Fmoc removal times for Fmoc-e-ctc and Fmoc-de-ctc that cause poor yield when synthesising HSSKLQ↓LGGde. Fmoc removal for all peptides without Asp residues were carried out by exposing the resin to 20% piperidine in DMF solutions (3×3 min, 9 mL total), except where shorter intervals were used to avoid cleavage from the resin for Fmoc-e-ctc (2×2 min, 1×1 min, 9 mL total). Fmoc removal from Fmoc-de-ctc was performed using 20% piperidine in DMF with 0.1 M Oxyma Pure for short intervals to decrease the formation of diketopiperizine and aspartimide (1×2 min, 1×1 min, 6 mL total). Fmoc removal from peptide sequences containing Asp residues was performed in 20% piperidine in DMF with 0.1 M Oxyma Pure.

For all steps, the extent of Fmoc removal was monitored using the Kaiser test, and except for the short time intervals specified above, the Fmoc removal step was repeated if the Kaiser test indicated incomplete removal. After Fmoc removal was complete, the resin was washed as detailed above.

To couple residues, the amino acid (4 eq.), PyBOP (4 eq.), and NMM (12 eq.) were combined in DMF to yield a 0.5 M solution with respect to the amino acid. All couplings were allowed to proceed for 45 min and performed in duplicate. The coupling solution was expelled, and the resin was washed. The extent of coupling was monitored using the Kaiser test, where a negative result for free amines indicated that coupling had fully proceeded.

### Coupling of Fluorescent Tags

When the sequences (HSSKLQ, HSSKLQL, and HSSKLQLGGde) had been synthesised on resin, the N-terminal Fmoc group was removed, then the resin was washed, and the coupling solution for the tags was added. Coupling of the AQ tag proceeded as for coupling an amino acid, while lower levels and longer intervals were used to couple the Re(I) complex tag (3 eq. Re(I) tag, 3 eq. PyBOP, 9 eq. NMM in DMF to make 0.5 M solutions; 24 hours).

Cleavage from Resins

Tag-peptide conjugates were cleaved from the resins and globally deprotected by shaking for 2.5 hours in 95% TFA, 2.5% TIS, and 2.5% water (2 mL). The cleavage solution was expelled, and the resin was exposed to neat TFA for 1hour. The cleavage solutions were combined and concentrated under a stream of nitrogen gas. Analysis by MALDI-TOF confirmed the presence of each conjugate and global deprotection of the AQ-peptides but showed that some tBu groups remained on the sidechains of Re-peptide conjugates. The Re-peptides were exposed to 90% TFA, 5% TIS, and 5% water for a further 1.5 hours to fully remove the tBu groups, confirmed by matrix-assisted laser desorption-ionisation time-of-flight (MALDI-TOF).

### Peptide Purification

Peptides were purified in the reverse phase to >95% purity using milliQ water and MeCN, buffered with 0.1% v/v TFA. Semi-preparative HPLC was performed using a Waters 2695 controller and pump, and a Waters Sunfire C18 column (OBD 5 μm, 10 mm×250 mm, at 4 mL/min).

Preparative HPLC was performed using a Waters 2535 Quaternary Gradient module, and a Waters Sunfire C18 column (OBD 5 μm, 19 mm×150 mm, at 7 mL/min). The UV traces were monitored at 254 nm, and peaks were collected using automated fraction collection systems.

AQ-HSSKLQ↓LGGde: preparative, 0-100% MeCN over 30 min, RT=17.4 min; AQ-HSSKLQ: semi-preparative, 0-50% MeCN over 30 min, RT=22.1 min; AQ-HSSKLQ↓L: preparative, 20-80% MeCN over 30 min, RT=12.8 min; Re-HSSKLQ↓LGGde: preparative, 30-40 MeCN over 30 min; RT=8.1 min; Re-HSSKLQ: preparative, 20-80% over 30 min, RT=15.3 min; Re-HSSKLQ↓L: preparative, 30-40% MeCN over 30 min, RT=8.3 min.

### Peptide Characterisation

Peptides were characterised by MALDI-TOF mass spectrometry and analytical HPLC. MALDI-TOF analysis was performed using a Bruker autoflex speed TOF in reflectron positive mode, with samples co-crystallised with an a-cyano-4-hydroxycinnamic acid matrix on steel plates. Reverse phase analytical HPLC was performed using a Waters 2695 controller and pump, and a Waters Sunfire C18 column (OBD 5 μm, 2.1 mm×150 mm, at 0.2 mL/min) with milliQ and MeCN buffered with 0.1% v/v TFA. Samples were dissolved in 1:1 milliQ:MeCN with 0.1% TFA and a concentration of guanidium hydrochloride to 1 M to discourage peptide folding. Analytical HPLC traces were obtained over a gradient of 0-100% MeCN over 30 min and were monitored at 254 nm.

AQ-HSSKLQ↓LGGde: yield 7.9 mg, 5.41 μmol, 4%, LRMS m/z 729 [M]², RT =16.5 min; AQ-HSSKLQ: yield 44.3 mg, 44.8 μmol, 68%, LRMS m/z 990.41 [M]⁺, RT =13.9 min; AQ-HSSKLQ↓L: yield 43.6 mg, 39.5 μmol, 54%, MS m/z 1103.52 [M]⁺, RT =17.4 min.

### PSA Cleavage Studies

Methods and conditions used for the PSA cleavage studies were based on those previously reported. Enzymatically active PSA purified from human seminal fluid was obtained from GenWay Biotech (0.59 mg/mL solution in 10 mM PBS, >95% purity by SDS-PAGE, pH 7.4 m 30 kDa, no preservatives present). AQ-HSSKLQ↓LGGde, AQ-HSSKLQ, and AQ-HSSKLQ↓L were dissolved in milliQ water to make 5 mM stock solutions. Liquid chromatography mass spectrometry (LCMS) traces in the reverse phase were obtained using a Shimadzu UFLC LCMS, including a CBM-20A controller, a DGU-20A3 degasser, two LC-20AD pumps, a CTO-20A column oven, an SPD-M20A photodiode array detector, and an LCMS-2020 mass spectrometer. Separation was achieved using a Waters Xbridge BEH130 C18 analytical column (OBD 5 μm, 4.6 mm×150 mm, at 0.2 mL/min) using milliQ water and MeCN each with 0.1% v/v formic acid buffer. PSA (200 μmol, 10 μL of 20 μM solution in PBS) was incubated with each substrate (40000 μmol, 8 μL of a 5 mM stock solution) at 37° C. in Tris-buffered saline (TBS) to a total volume of 200 μL. For compounds AQ-HSSKLQ↓LGGde, AQ-HSSKLQ, and AQ-HSSKLQ↓L, 20 μL aliquots of the incubated solution were collected once at 3- and 6-hours' incubation and were collected in triplicate at 24- and 48-hours' incubation.

Each aliquot was made up to 100 μL with a 50% MeCN in milliQ solution and was centrifuged then syringe filtered. A 20 μL sample was injected onto the LCMS and analysed over a range of m/z 200-2000, over a gradient of 0-100% MeCN in milliQ water, over 60 min. Component compounds that eluted over the course of the gradient were monitored at 254 nm. Control incubations containing only substrate in buffer, or only PSA in buffer, were also performed and analysed in the same manner to confirm the stability of the conjugates in the experimental conditions, and that cleavage of the Gln-Leu bond in AQ-HSSKLQ↓LGGde was PSA-mediated.

### Cell Lines and Maintenance

Human-derived colorectal cancer DLD-1 cells (a colorectal adenocarcinoma cell line) were obtained from American Type Culture Collection and were used from passages 6-20 within 3 months of resuscitation. Cells were maintained in exponential growth in Adv DMEM, supplemented with 2 mM glutamine and 2% FBS, and in a humidified environment with 5% CO₂. Cells were seeded into a 96-well plate with 100 μL of Adv DMEM, supplemented with 2% FBS and 2 mM glutamine. The numbers of cells seeded per well, and the plate material (glass or plastic) are listed in the cell image figure captions. Cells were allowed to adhere overnight under standard incubation conditions. Each experimental condition was performed in triplicate. Prior to dosing, the cells were gently washed with Adv DMEM supplemented with 2 mM glutamine (3×100 μL). Cells were dosed in Adv DMEM supplemented with 2 mM glutamine, with a final substrate concentration of 50 μM unless otherwise specified. Cells were dosed either without PSA, or with added PSA (2 μg, 67 μmol, yielding a final concentration of 20 μg/mL unless otherwise specified). Plates were incubated under standard conditions.

Prior to imaging, the media was removed from each well, and the cells were gently washed with PSA (3×100 μL). Fluorobrite DMEM supplemented with 2 mM glutamine (100 μL) was added to each well prior to imaging at specified time points.

### Confocal Fluorescence Microscopy

Confocal fluorescence microscopy was performed using an Olympus FV3000 inverted fluorescence microscope equipped with a UPLSAPO 10× dry objective lens (N.A.=4.0), and a heat-controlled stage maintained at 37° C., supplied with 5% CO₂. The AQ peptide conjugates were excited using a 561 nm LED laser, and emission was monitored over a range of 570-670 nm. All images were acquired using line averaging (3 times per line), a scan rate of 2 μs per pixel, and a digital zoom of 5.0. Three different locations within each well were chosen for imaging. Micrographs were imaged in which cells were at the best focus on brightfield. Images were acquired using Olympus FV31S-SW-v2.4.1.198 software. The 561 nm laser was used at 50% power, 650 V, 1× gain, and 3% offset. Scale bars represent 30 μm. Images were processed using Fiji ImageJ v1.53c. All fluorescence images were processed using a range of 20-300 in the brightness and contrast settings unless otherwise specified.

### REFERENCES

1. Di Marco, L. et al. “Modulating the Cellular Uptake of Fluorescently Tagged Substrates of Prostate-Specific Antigen before and after Enzymatic Activation”, Bioconjugate Chemistry 30, 124-133, doi: 10.1021/acs.bioconjchem.8b00792 (2019).
2. Eissler, S. et al. “Substitution determination of Fmoc-substituted resins at different wavelengths”, Journal of Peptide Science 23, 757-762 (2017).
3. Feng, Z. & Xu, B. “Inspiration from the mirror: D-amino acid containing peptides in biomedical approaches”, Biomol Concepts 7, 179-187, (2016).
4. Frenette, G., Gervais, Y., Tremblay, R. R. & Dube, J. Y. “Contamination of purified prostate-specific antigen preparations by kallikrein hK2”, The Journal of Urology 159, 1375-137, (1998).
5. Green, B. P. “Matrix metalloproteinase binding agents for luminescence and radioimaging of metastatic tumours”, Doctor of Philosophy thesis, The University of Sydney, (2013).
6. Greene, R. F. & Pace, C. N. “Urea and guanidine hydrochloride denaturation of ribonuclease, lysozyme, a-chymotrypsin, and βlactoglobulin”, Journal of Biological Chemistry 249, 5388-5393 (1974).
7. Hortobagyi, G. “Anthracyclines in the treatment of cancer”, Drugs 54, 1-7 (1997).
8. Kaiser, E., Colescott, R., Bossinger, C. & Cook, P. “Color test for detection of free terminal amino groups in the solid-phase synthesis of peptides”, Analytical Biochemistry 34, 595-598 (1970).
9. Kizek, R. et al. “Anthracyclines and ellipticines as {DNA}-damaging anticancer drugs: Recent advances. Pharmacology & Therapeutics”, 133, 26-39 (2012).
10. Lazarova, N., James, S., Babich, J. & Zubieta, J. “A convenient synthesis, chemical characterization and reactivity of [Re(CO)₃(H₂O)₃]Br: the crystal and molecular structure of [Re(CO)₃(H₂O)₃]Br”, Inorganic Chemistry Communications 7, 1023-1026 (2004).
11. Liu, M. et al. “D-Peptides as Recognition Molecules and Therapeutic Agents”, The Chemical Record 16, 1772-1786, (2016).
12. Menez, R. et al. “Crystal Structure of a Ternary Complex between Human Prostate-specific Antigen, Its Substrate Acyl Intermediate and an Activating Antibody”, Journal of Molecular Biology 376, 1021-1033 (2008).
13. Mergler, M., Dick, F., Sax, B., Weiler, P. & Vorherr, T. “The aspartimide problem in Fmoc-based SPPS” Part I. Journal of Peptide Science 9, 36-46, doi: doi: 10.1002/psc.430 (2003).
14. Remington's Pharmaceutical Science, 18th ed., Mack Publishing Company, Easton, Pa. (1990).
15. Remington: the Science and Practice of Pharmacy, 20th ed., Lippincott Williams & Wilkins, (2003).
16. Theoretical and Computational Biophysics Group, www.ks.uiuc.edu, www.ks.uiuc.edu (March 2006, Accessed 2018).

### Conclusion

This work involved the design and synthesis of model prodrugs to target prostate cancer, incorporating a pro-moiety with a peptide sequence that is cleaved with high selectivity by prostate specific antigen (PSA), a negatively charged sequence for both electrostatic interaction with the arginine patch of PSA and to slow cellular uptake, and a fluorescent tag to allow imaging of the in vitro distribution of the model prodrugs.

The results described herein provide that negatively charged peptide sequences, including lipophilic spacers, can be readily modelled and designed in the active site of PSA using computational molecular simulations. Modelled interactions between HSSKLQ↓LGGde and PSA showed the desired electrostatic interactions between the negatively charged sequence and the arginine patch of PSA.

Further, these studies showed that amino acids with D chirality could be readily incorporated in the models, and that they did not incur an energy penalty compared with corresponding sequences with L chirality.

Despite the limitations of the AQ-peptide conjugates in these studies, these results demonstrate resistance to uptake by cells in the absence of PSA. The model prodrug does not appear to be vulnerable to proteolytic degradation in this in vitro model, likely due to the incorporation of the C-terminal D-Asp-D-Glu negatively charged sequence. Moreover, the prodrug is activated by PSA in vitro, even at concentrations of PSA that are 1000-fold lower than that found in the extracellular fluid of prostate cells.

### Advantages

The pro-moiety and composition comprising the pro-moiety for use in forming a prodrug for detecting and/or treating prostate cancer, as described in embodiments of the present invention herein, provide a number of advantages, including, but not limited to:
the pro-moiety of the prodrug has a selectively cleavable peptide sequence in the presence of prostate specific antigen (PSA) to yield a cleaved peptide-drug conjugate;
the cleaved peptide-drug conjugate is expected to be taken up by cells in the immediate environment;
the resulting prodrug produced from the pro-moiety described herein is selective towards prostate cancer.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

### Other Embodiments

It will be understood by persons of skill in the relevant art that the model prodrug and composition thereof as described in embodiments of the present invention above, are not simply limited to a conjugate formed between the peptide sequence and the anthraquinone (AQ)-type drugs described above, but may be adapted to be conjugated to other drugs for detecting and/or treating prostate cancer, including the drugs approved for prostate cancer by, for example, the NIH National Cancer Institute (see https://www.cancer.gov/about-cancer/treatment/drugs/prostate).

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used.

Although the invention has been described with reference to specific examples, it will be appreciated by those skilled in the art that the invention may be embodied in many other forms.

### Industrial Applicability

It is apparent from the above, that the arrangements described are applicable to the medical and healthcare industry.

1. A pro-moiety, comprising:
a first peptide; and
a second peptide that is linked to the first peptide,
wherein the first peptide comprises a sequence that is configured near a first terminus for conjugating to a drug to form a prodrug that is rapidly and/or highly selectively cleaved by prostate-specific antigen (PSA), and configured at a second terminus to bind with high selectivity to the active site of PSA, and the second peptide comprises a sequence having a negative charge to slow uptake of the prodrug by cells, and
wherein the second peptide is cleaved from the first peptide upon proteolysis by PSA to produce a conjugate of the first peptide and the drug that is suitable for uptake by target cells.

2. A composition comprising a pro-moiety, the pro-moiety comprising:
a first peptide; and
a second peptide that is linked to the first peptide,
wherein the first peptide comprises a sequence that is configured near a first terminus for conjugating to a drug to form a prodrug that is rapidly and/or highly selectively cleaved by prostate-specific antigen (PSA), and configured at a second terminus to bind with high selectivity to the active site of PSA, and the second peptide comprises a sequence having a negative charge to slow uptake of the prodrug by cells, and
wherein the second peptide is cleaved from the first peptide upon proteolysis by PSA to produce a conjugate of the first peptide and the drug that is suitable for uptake by target cells.

3. The pro-moiety of claim 1, wherein the first peptide sequence comprises HisSerSerLysLeuGln (HSSKLQ).

4. The pro-moiety of claim 1, wherein the first terminus of the first peptide is the N-terminus.

5. The pro-moiety of claim 1, wherein the second peptide comprises one or more amino acids that is negatively charged at physiological pH.

6. The pro-moiety of claim 1, wherein the second peptide includes the sequence D-Asp-D-Glu (de).

7. The pro-moiety of claim 1, wherein the second peptide is linked to the C-terminus of the first peptide via a spacer.

8. The pro-moiety of claim 7, wherein the spacer is an amino acid sequence, and wherein a first amino acid of the spacer is compatible with the active site of PSA.

9. The pro-moiety of claim 8, wherein the first amino acid of the spacer is a leucine residue.

10. The pro-moiety of claim 8, wherein the spacer comprises the amino acid sequence LeuGlyGly (LGG).

11. A method for detecting and/or treating prostate cancer in a subject, the method comprising administering to the subject, a therapeutically or diagnostically effective amount of a composition according to claim 2.

12. The method of claim 11, wherein the composition is administered intratumorally and/or intraprostatically.

13. The method of claim 11, wherein the composition is administered intravenously, intramuscularly, subcutaneously.

14. The method of claim 11, wherein the subject has a localized prostate tumour.

15. The method of claim 11, wherein the subject has a metastatic prostate tumour.

16. The method of claim 11, wherein administration results in a reduction in prostate tumour volume.

17. The method of claim 11, wherein administration results in a reduction of a metastatic prostate tumour.

18. The method of claim 17, wherein administration results in treatment of the metastatic prostate tumour.

19. (canceled)

20. The composition of claim 2, wherein the first peptide sequence comprises HisSerSerLysLeuGln (HSSKLQ).


SEQUENCE LISTING (30 sequences)

SEQ ID NO: 1 - protein, 6 aa, synthetic construct
HSSKLQ

SEQ ID NO: 2 - protein, 11 aa, synthetic construct
  SITE 6..7: note = Location of Prostate Specific Antigen (PSA) cleavage (the PSA scissile bond); between residues 6 and 7
  SITE 10: note = D-Aspartic Acid
  SITE 11: note = D-Glutamic Acid
HSSKLQLGGDE

SEQ ID NO: 3 - protein, 6 aa, synthetic construct
HSSKLQ

SEQ ID NO: 4 - protein, 5 aa, synthetic construct
  SITE 4: note = D-Aspartic Acid
  SITE 5: note = D-Glutamic Acid
LGGDE

SEQ ID NO: 5 - protein, 11 aa, synthetic construct
  SITE <1: note = Fluorescent Anthraquinone (AQ) tag
  SITE 6..7: note = Location of Prostate Specific Antigen (PSA) cleavage (the PSA scissile bond); between residues 6 and 7
  SITE 10: note = D-Aspartic Acid
  SITE 11: note = D-Glutamic Acid
HSSKLQLGGDE

SEQ ID NO: 6 - protein, 6 aa, synthetic construct
  SITE <1: note = Fluorescent Anthraquinone (AQ) tag
HSSKLQ

SEQ ID NO: 7 - protein, 7 aa, synthetic construct
KGISSQY

SEQ ID NO: 8 - protein, 4 aa, synthetic construct
LDDD

SEQ ID NO: 9 - protein, 5 aa, synthetic construct
LDDDD

SEQ ID NO: 10 - protein, 4 aa, synthetic construct
LEEE

SEQ ID NO: 11 - protein, 5 aa, synthetic construct
LEEEE

SEQ ID NO: 12 - protein, 5 aa, synthetic construct
LGDDD

SEQ ID NO: 13 - protein, 5 aa, synthetic construct
SGDDD

SEQ ID NO: 14 - protein, 5 aa, synthetic construct
LGEEE

SEQ ID NO: 15 - protein, 5 aa, synthetic construct
SGEEE

SEQ ID NO: 16 - protein, 5 aa, synthetic construct
  SITE 3..5: note = D-Aspartic Acid
LGDDD

SEQ ID NO: 17 - protein, 5 aa, synthetic construct
  SITE 3..5: note = D-Aspartic Acid
SGDDD

SEQ ID NO: 18 - protein, 5 aa, synthetic construct
  SITE 3..5: note = D-Glutamic Acid
LGEEE

SEQ ID NO: 19 - protein, 5 aa, synthetic construct
  SITE 3..5: note = D-Glutamic Acid
SGEEE

SEQ ID NO: 20 - protein, 5 aa, synthetic construct
  SITE 4: note = D-Aspartic Acid
  SITE 5: note = D-Glutamic Acid
LGGDE

SEQ ID NO: 21 - protein, 5 aa, synthetic construct
  SITE 4: note = D-Aspartic Acid
  SITE 5: note = D-Glutamic Acid
SGGDE

SEQ ID NO: 22 - protein, 5 aa, synthetic construct
LGGDE

SEQ ID NO: 23 - protein, 5 aa, synthetic construct
SGGDE

SEQ ID NO: 24 - protein, 7 aa, synthetic construct
HSSKLQL

SEQ ID NO: 25 - protein, 7 aa, synthetic construct
  SITE <1: note = Fluorescent Anthraquinone (AQ) tag
  SITE 6..7: note = Location of Prostate Specific Antigen (PSA) cleavage (the PSA scissile bond); between residues 6 and 7
HSSKLQL

SEQ ID NO: 26 - protein, 11 aa, synthetic construct
  SITE <1: note = Luminescent Rhenium(I) Complex tag
  SITE 6..7: note = Location of Prostate Specific Antigen (PSA) cleavage (the PSA scissile bond); between residues 6 and 7
  SITE 10: note = D-Aspartic Acid
  SITE 11: note = D-Glutamic Acid
HSSKLQLGGDE

SEQ ID NO: 27 - protein, 6 aa, synthetic construct
  SITE <1: note = Luminescent Rhenium(I) Complex tag
HSSKLQ

SEQ ID NO: 28 - protein, 7 aa, synthetic construct
  SITE 6..7: note = Location of Prostate Specific Antigen (PSA) cleavage (the PSA scissile bond); between residues 6 and 7
  SITE <1: note = Location of Prostate Specific Antigen (PSA) cleavage (the PSA scissile bond); between residues 6 and 7
HSSKLQL

SEQ ID NO: 29 - protein, 11 aa, synthetic construct
  SITE <1: note = Tag that is either Fluorescent Anthraquinone (AQ) tag or Luminescent Rhenium(I) Complex tag
  SITE 6..7: note = Location of Prostate Specific Antigen (PSA) cleavage (the PSA scissile bond); between residues 6 and 7
  SITE 10: note = D-Aspartic Acid
  SITE 11: note = D-Glutamic Acid
HSSKLQLGGDE

SEQ ID NO: 30 - protein, 6 aa, synthetic construct
  SITE <1: note = Tag that is either Fluorescent Anthraquinone (AQ) tag or Luminescent Rhenium(I) Complex tag
HSSKLQ